(19) United States
(12) Patent Application Publication
(10) Pub. No.: US 2012/0070397 A1
(43) Pub. Date: Mar. 22, 2012

(54) MAINTAINING FEET DRY TO PREVENT FREEZING

(76) Inventor: Scotty Wales, Vidor, TX (US)

(21) Appl. No.: 12/884,512

(22) Filed: Sep. 17, 2010

(51) Int. Cl. A61K 8/28; A61Q 15/00; A61K 8/58; A61K 8/69; A61K 8/26; A61K 8/31

(52) U.S. Cl. 424/66; 424/65; 424/68

(57) ABSTRACT

A method of keeping the feet warm when engaging in activities in cold weather, comprising the step of applying antiperspirant to the feet. This is done prior to donning foot coverings such as socks. The foot coverings may comprise a wicking fabric or wool. The antiperspirant may be a liquid based product including aluminum chlorohydrate, or a solid type product including aluminum chlorohydrate or aluminum zirconium tetrachlorohydrox GLY.

### FIELD OF THE INVENTION

The present invention relates to treating the feet to keep the feet dry, thereby preventing undesired heat transfer to footwear and to the environment in cold weather.

### BACKGROUND OF THE INVENTION

Keeping the feet warm in cold weather has long plagued the human race. Activities which are conducted in cold weather may include hunting, fishing, hiking, skiing, and others. The usual response of people engaging in such activities to cold weather is to increase the thickness or number of foot coverings or both. While this can slow heat loss to the ambient in the short run, it can simultaneously promote conditions which in the longer term actually facilitate heat loss or which also induce new problems.

Increasing insulation layers around the feet helps keep the feet warm in the short term. However, the feet may become so hot as to promote perspiration. Perspiration can build up within the footwear and eventually provide a heat transfer path which initially may restore comfort by conducting excess heat away, but which if continued, may undesirably and excessively transfer too much heat to the environment. When this happens, the feet can freeze despite the presence of thermally insulating layers because those layers have been rendered ineffective due to conduction of heat through the moisture of the perspiration.

It should also be noted that when the layers of potentially warming insulation build up, they increase pressure within the shoe, which can cause constriction of blood vessels, with consequent reduction in the supply of heated blood to the foot. Paradoxically, then, the attempt to maintain warmth at the foot by increasing the layers of coverings may provoke a counter-effect which again may lead to objectionable cooling of the feet.

There exists a need for a method of keeping feet warm but not excessively so when a person encounters cold weather.

### SUMMARY OF THE INVENTION

The present invention provides a method of preventing perspiration at the feet in cold weather. The method is that of preemptively applying antiperspirant to the foot. More particularly, the antiperspirant may be applied to the sole and between the soles. Antiperspirant may be applied as a liquid or as a solid. Components of an antiperspirant usable in the present invention may include aluminum chlorohydrate used with liquids for example, and aluminum zirconium tetrachlorohydrox GLY with solid compositions. Application of antiperspirant may be enhanced by also wearing socks or equivalent garments which comprise wicking fabrics and wool.

It is an object of the invention to provide a method of overcoming the drawbacks of prior art methods of keeping the feet warm when engaging in activities in very cold weather.

Another object of the invention is to prevent loss of heat due to perspiration of the feet.

It is an object of the invention to provide improved elements and arrangements thereof by apparatus for the purposes described which is inexpensive, dependable, and fully effective in accomplishing its intended purposes.

### DETAILED DESCRIPTION

According to at least one aspect, the present invention is a method of keeping feet warm when engaging in activities in cold environments. Cold environments may include the outdoors, such as is the case when hunting, fishing, trapping, hiking, conducting surveillance, engaging in photography, hiking, skiing, participating in an activity as a spectator, and the like. Cold environments may also include indoor environments, such as walk-in freezers, cryogenic facilities, and others. The method will be understood to supplement conventional steps such as donning of one or more layers of socks, insulated footwear such as boots, and the like. At the core of the invention lies the step of applying an antiperspirant to the feet prior to donning the foot apparel. This is done proactively, while the feet are dry. The feet may be dried with a towel for example to assure dryness.

The method may comprise a further step of donning socks or other foot coverings comprising a wicking fabric or material or of wool, or donning socks or other foot coverings comprising both a wicking fabric material and also wool.

The selected antiperspirant may be a liquid, such as a liquid comprising aluminum chlorohydrate. The aluminum chlorohydrate may be limited 8.2 percent of the antiperspirant. The liquid may comprise at least one of the components including butane, cyclopentasiloxane, hydrofluorocarbon 152a, talc, isobutane, and silica dimethylsilylate. Regardless of other components, the antiperspirant may exclude or be devoid of any substance which is conventionally used to impart fragrance.

An antiperspirant in liquid form may comprise aluminum zirconium tetrachlorohydrox GLY and at least one of water and glycerine.

Alternatively, the antiperspirant may take solid form. As employed herein, solid need not imply rigidity. Rather, the term “solid” should be interpreted in accordance with antiperspirant products presently available to the public on a commercial basis. The antiperspirant, if solid, may comprise aluminum zirconium tetrachlorohydrox GLY, which may be limited to 19 percent of the antiperspirant.

The present invention is susceptible to modifications and variations which may be introduced thereto without departing from the inventive concepts. For example, a suitable antiperspirant composition may contain both aluminum chlorohydrate and also aluminum zirconium tetrachlorohydrox GLY. Further, other active ingredients having antiperspirant qualities may be used in addition to either of or both aluminum chlorohydrate and also aluminum zirconium tetrachlorohydrox GLY.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A method of keeping feet warm when engaging in activities in cold environments, comprising the step of applying an antiperspirant to the feet prior to donning foot apparel.

2. The method of claim 1, wherein the antiperspirant is a liquid.

3. The method of claim 2, wherein the liquid comprises aluminum chlorohydrate.

4. The method of claim 3, wherein the liquid further comprises at least one of the components including butane, cyclopentasiloxane, hydrofluorocarbon 152a, talc, isobutane, and silica dimethylsilylate.

5. The method of claim 4, wherein the antiperspirant is devoid of a substance used to impart fragrance.

6. The method of claim 4, wherein the aluminum chlorohydrate is limited to 8.2 percent of the antiperspirant.

7. The method of claim 2, wherein the liquid comprises aluminum zirconium tetrachlorohydrox GLY and at least one of water and glycerine.

8. The method of claim 1, wherein the antiperspirant is a solid.

9. The method of claim 8, wherein the solid comprises aluminum zirconium tetrachlorohydrox GLY.

10. The method of claim 9, wherein the aluminum zirconium tetrachlorohydrox GLY is limited to 19 percent of the antiperspirant.

11. The method of claim 1, comprising the further step of donning a foot covering comprising a wicking material.

12. The method of claim 1, comprising the further step of donning a foot covering comprising wool.
